(19) United States
(12) Patent Application Publication
(10) Pub. No.: US 2022/0401934 A1
(43) Pub. Date: Dec. 22, 2022

(54) METHOD OF IMPROVING ISOMERIZATION CATALYST LIFETIME

(71) Applicant: Lyondell Chemical Technology, L.P., Houston, TX (US)

(72) Inventors: Rick B. Watson, Houston, TX (US); David W. Leyshon, Houston, TX (US); Kevin P. Fogarty, Houston, TX (US)

(73) Assignee: Lyondell Chemical Technology, L.P., Houston, TX (US)

(21) Appl. No.: 17/831,563

(22) Filed: Jun. 3, 2022

Related U.S. Application Data
(60) Provisional application No. 63/208,871, filed on Jun. 9, 2021.

(51) Int. Cl. B01J 29/67; C07C 5/25

(52) U.S. Cl. CPC B01J 29/67; C07C 5/2518; C07C 2529/67

(57) ABSTRACT

A skeletal isomerization process for isomerizing olefins is described. The process utilizes added hydrogen as a diluent to extend the isomerization catalyst's lifetime and increase the yield of skeletal isomer products compared to process that utilize inert gas diluents. The methods of this disclosure can be applied to feeds containing iso-olefins (for the production of linear olefins) or linear olefins (for the production of iso-olefins).

### PRIOR RELATED APPLICATIONS

This application claims the benefit of priority to U.S. Provisional Patent Application No. 63/208,871, filed on Jun. 9, 2021, which is incorporated herein by reference in its entirety.

### FEDERALLY SPONSORED RESEARCH STATEMENT

Not applicable.

### FIELD OF THE DISCLOSURE

The disclosure generally relates to skeletal isomerization processes, and more specifically to a method of improving the lifetime of the catalyst used in an olefin skeletal isomerization process.

### BACKGROUND OF THE DISCLOSURE

Zeolite materials, both natural and synthetic, are known to have catalytic properties for many industrially relevant chemical reactions. Zeolites are ordered porous crystalline aluminosilicates having a definite structure with cavities interconnected by channels. The cavities and channels throughout the crystalline material can be of such a size to allow selective reaction of hydrocarbons. Such hydrocarbon reactions by the crystalline aluminosilicates essentially depends on discrimination between molecular dimensions. Consequently, these materials in many instances are known in the art as “molecular sieves” and are used, in addition to catalytic properties, for certain selective adsorptive processes.

In many instances, it is desirable to convert a methyl branched olefin such as isobutylene, to a linear olefin, such as 1-butene, by mechanisms such as skeletal isomerization. EP Patent No. 0523838 (Lyondell) describes a process of skeletal isomerization of linear olefins, or iso-olefins, with a catalyst of zeolite type for converting the linear olefins to iso-olefins, or vice versa.

However, during the isomerization process, a portion of the olefin molecules aggregate at or in the channels of the zeolite catalyst, on adjacent active sites, resulting in dimerization or oligomerization that lead to byproducts of longer chains and heavier molecular weights than the desired product. Consequently, the yield and conversion rate of the desired product is reduced, particularly in the beginning hours of the time-on-stream. During this period of unselective transformation, coke is deposited on the catalyst surface and the yield of beneficial products increases with time while that of undesired products decreases. The coke deposited in this initial period of time is deemed beneficial in that it diminished the unselective transformations. However, the coking and production of heavier byproducts also increase the rate of deactivation of the zeolite catalyst, thus reducing its lifetime.

There still exists a need for an economical process that can increasing the yield and conversion rate to the isomerization product while reducing the deactivation rate of the catalyst.

### SUMMARY OF THE DISCLOSURE

The present disclosure is directed to novel methods for structurally isomerizing hydrocarbon streams containing one or more olefins. In particular, a skeletal isomerization process that utilizes added hydrogen as a diluent is disclosed. The addition of an inert gas diluent such as helium, argon, nitrogen, or saturated hydrocarbons such as methane or n-butane has been known to extend the catalyst lifetime for certain catalysts and reactions simply by a reduction in the concentration of species that may lead to deactivation. However, the addition of hydrogen was surprisingly found to be even better at extending the catalyst lifetime, regardless of the zeolite being used. The use of hydrogen also results in an increase in the yield of skeletal isomer products for a longer period of time.

The present methods include any of the following embodiments in any combination(s) of one or more thereof:

A skeletal isomerization process comprising the steps of co-feeding a hydrocarbon feed comprising at least one olefin and a hydrogen feed to a reactor containing an isomerization zeolite catalyst; and, isomerizing at least one olefin to a skeletal isomer product in the reactor for at least one catalyst cycle.

A skeletal isomerization process comprising the steps of co-feeding a hydrocarbon feed comprising at least one olefin and a hydrogen feed to a reactor containing an isomerization zeolite catalyst, wherein the hydrocarbon feed is fed at a weight hourly space velocity (WHSV) between 1 to 30 h⁻¹; and isomerizing the at least one olefin to at least one skeletal isomer product in the reactor for at least one catalyst cycle, wherein the catalyst cycle is at least sixteen days.

A skeletal isomerization process comprising the steps of co-feeding a hydrocarbon feed comprising at least one olefin and a hydrogen feed to a reactor containing an isomerization zeolite catalyst, wherein the hydrocarbon feed is fed at a weight hourly space velocity (WHSV) between 1 to 30 h⁻¹ and the molar ratio of the hydrocarbon feed to the hydrogen feed is between about 1:0.01 to about 1:1; and isomerizing the at least one olefin to at least one skeletal isomer product in the reactor for at least one catalyst cycle, wherein the catalyst cycle is at least sixteen days, the temperature of the reactor is from about 340° C. to about 500° C., and the isomerization zeolite catalyst is the hydrogen form of ferrierite (H-FER).

Any of the processes described herein, further comprising the step of recovering the skeletal isomer product from the reactor.

Any of the processes described herein, wherein the skeletal isomer product comprises 1-butene and 2-butene.

Any of the processes described herein, wherein the skeletal isomer product comprises isobutylene.

Any of the processes described herein, wherein the at least one olefin is a linear olefin.

Any of the processes described herein, wherein the at least one olefin is 1-butene and 2-butene.

Any of the processes described herein, wherein the at least one olefin is isobutylene.

Any of the processes described herein, wherein the hydrocarbon feed comprises at least 40 wt. % isobutylene.

Any of the processes described herein, wherein the hydrocarbon feed further comprises alkanes, aromatics, hydrogen and other gases.

Any of the processes described herein, wherein in the at least one olefin is isobutylene and the at least one skeletal isomer product is 1-butene and 2-butene.

Any of the processes described herein, wherein in the at least one olefin comprises 1-butene and 2-butene, and the at least one skeletal isomer product is isobutylene.

Any of the processes described herein, wherein the molar ratio of at least one olefin in the hydrocarbon feed to the hydrogen feed during the co-feeding step is between about 1:0.01 to about 1:1.

Any of the processes described herein, wherein the molar ratio of the hydrocarbon feed to the hydrogen feed during the co-feeding step is between about 1:0.01 to about 1:1, or wherein the ratio of the hydrocarbon feed to the hydrogen feed is between about 2.5 vol. % and up to 50 vol. %, based on the volume of the total feed.

Any of the processes described herein, wherein the temperature of the reactor is between about 340° C. to 500° C.

Any of the processes described herein, wherein the temperature of the reactor is between about 380° C. to 425° C.

Any of the processes described herein, wherein the pressure of the reactor is between about zero to about 345 kPa (50 psig).

Any of the processes described herein, wherein the hydrocarbon weight hour space velocity (WHSV) of the hydrocarbon feed is in the range of from about 1 to about 3011¹ (1 to 30 g isobutylene/g catalyst/h).

Any of the processes described herein, wherein the hydrocarbon weight hour space velocity (WHSV) of the hydrocarbon feed is in the range of from about 1 to about 1011¹ (1 to 10 g isobutylene/g catalyst/h)

Any of the processes described herein, wherein the catalyst cycle is at least sixteen days, at least 21 days, or at least 25 days in length when the WHSV is 211¹.

Any of the processes described herein, wherein the catalyst cycle is at least sixteen days when the WHSV is 2

Any of the processes described herein, wherein the isomerization zeolite catalyst is hydrogen form of ferrierite (H-FER).

Any of the processes described herein, wherein the isomerization zeolite catalyst additionally comprises a binder material selected from the group consisting of: silica, silica-alumina, bentonite, kaolin, bentonite with alumina, montmorillonite, attapulgite, titania and zirconia.

Any of the processes described herein, wherein the isomerization zeolite catalyst has a silica to alumina ratio from 10:1 to 100:1.

Any of the processes described herein, wherein the ratio of time on stream for the at least one olefin conversion to reach 45% to linear butene (nB) yield is greater than 5.5:1 at an olefin feed weight hourly space velocity (WHSV) of 2 (g olefin/g catalyst/hr).

Any of the processes described herein, wherein the ratio of time on stream for the at least one olefin conversion to reach 45% to linear butene (nB) yield is greater than 4.5:1 at an olefin feed weight hourly space velocity of 3 (g olefin/g catalyst/hr).

Any of the processes described herein, wherein the ratio of time on stream for the at least one olefin conversion to reach 45% to linear butene (nB) yield is greater than 2.75:1 at an olefin feed weight hourly space velocity of 5 (g olefin/g catalyst/hr).

Any of the processes described herein, wherein the ratio of time on stream for the at least one olefin conversion to reach 45% to linear butene (nB) yield is greater than 3.0:1 at an olefin feed weight hourly space velocity of 7 (g olefin/g catalyst/hr).

This summary is provided to introduce a selection of concepts that are further described below in the detailed description. This summary is not intended to identify key or essential features of the claimed subject matter, nor is it intended to be used as an aid in limiting the scope of the claimed subject matter.

### Definitions

As used herein, the term “skeletal isomerization” is used to refer to an isomerization process that involves the movement of a carbon atom to a new location on the skeleton of the molecule, e.g., from a branched isobutylene skeleton to a linear or straight chain (not branched) butene skeleton. The product in the skeletal isomerization process is a skeletal isomer of the reactant. The term “skeletal isomer” refers to molecules that have the same number of atoms of each element and the same functional groups, but differ from each other in the connectivity of the carbon skeleton.

As used herein, the term “zeolite” includes a wide variety of both natural and synthetic positive ion-containing crystalline aluminosilicate materials, including molecular sieves. Zeolites are characterized as crystalline aluminosilicates which comprise networks of SiO₄ and AlO₄ tetrahedra in which silicon and aluminum atoms are cross-linked in a three-dimensional framework by sharing of oxygen atoms. This framework structure contains channels or interconnected voids that are occupied by cations, such as sodium, potassium, ammonium, hydrogen, magnesium, calcium, and water molecules. The water may be removed reversibly, such as by heating, which leaves a crystalline host structure available for catalytic activity. The term “zeolite” in this specification is not limited to crystalline aluminosilicates. The term as used herein also includes silicoaluminophosphates (SAPO), metal integrated aluminophosphates (MeAPO and ELAPO), metal integrated silicoaluminophosphates (MeAPSO and ELAPSO). The MeAPO, MeAPSO, ELAPO, and ELAPSO families have additional elements included in their framework. For example, Me represents the elements Co, Fe, Mg, Mn, or Zn, and El represents the elements Li, Be, Ga, Ge, As, or Ti. An alternative definition would be “zeolitic type molecular sieve” to encompass the materials useful for this disclosure.

As used herein, “channel size” refers to the size of the channels in the zeolite structure and should not be confused with “crystal size” (the diameter of the zeolite crystals which exist in a zeolite catalyst) or “pore size” (the size of the pore, or opening, in the zeolite structure).

As used herein, “H-FER” or “hydrogen form of ferrierite” refers to a hydrogen exchanged ferrierite zeolite.

As used herein, the term “coke” refers to the formation of carbonaceous materials on a catalyst surface, particularly inside and around the mouths of channels. As understood in the field, coke is the end product of carbon disproportionation, condensation and hydrogen abstraction reactions of adsorbed carbon-containing material.

As used herein, the terms “decoking” and “catalyst regeneration” refers to the removal of coke from a catalyst's surface. While there are many ways for removing coke from a catalyst, one such method includes reactions of atomic oxygen with “coke” and yields gases such as CO, CO₂ as well as other gaseous products that could be removed.

As used herein, the terms “life cycle of the catalyst”, “catalyst cycle” or “catalyst lifetime” are used interchangeably to refer to the length of time the catalyst is in use before being regenerated.

As used herein, the term “unselective site” refers to an active site on the catalyst that catalyzes undesirable side reactions.

As used herein, “olefin” refers to any alkene compound that is made up of hydrogen and carbon that contains one or more pairs of carbon atoms linked by a double bond. A “C” followed by a number, in reference to an olefin, refers to how many carbon atoms the olefin contains. For example, a C4 olefin can refer to butene, butadiene, or isobutene. A plus sign (+) is used herein to denote a composition of hydrocarbons with the specified number of carbon atoms plus all heavier components. As an example, a C4+ stream comprises hydrocarbons with 4 carbon atoms plus hydrocarbons having 5 or more carbon atoms.

As used herein, WHSV or “weight hour space velocity” refers to the weight of hydrocarbon feed flowing per hour per unit weight of the catalyst. For example, for every 1 gram of catalyst, if the weight of hydrocarbon feed flowing is 100 grams per hour, then the WHSV is 100 H⁻¹.

As used herein, “atmosphere” in the context of pressure refers to 101,325 Pascal, or 760 mmHg, or 14.696 psi.

The terms “heavy olefins” is used to denote compositions of C5+ hydrocarbons, including mono-olefins and diolefins.

The term “conversion” is used to denote the percentage of a component fed which disappears across a reactor.

The term “2-butene” as used herein refers to both cis-2-butene and trans-2-butene.

The term “linear C4 olefin” or “normal butene” are used interchangeably herein to refer to 1-butene, cis-2-butene and/or trans-2-butene.

The term “normal butene yield” refers to the amount of normal, linear butenes, including 1- and 2-butene, formed during an isomerization process.

As used herein, the term “raffinate” refers to a residual stream of olefins obtained after the desired chemicals/material have been removed. In the cracking/crude oil refining process, a butene or “C4” raffinate stream refers to the mixed 4-carbon olefin stream recovered from the cracker/fluid catalytic cracking unit. The term “Raffinate 1” refers to a C4 residual olefin stream obtained after separation of butadiene (BD) from the initial C4 raffinate stream. “Raffinate 2” refers to the C4 residual olefin stream obtained after separation of both BD and isobutylene from the initial C4 raffinate stream. “Raffinate 3” refers to the C4 residual olefin stream obtained after separation of BD, isobutylene, and 1-butene from the initial C4 raffinate stream. In some embodiments of the present disclosure, the isobutylene separated from Raffinate 1 can be used as a source for the skeletal isomerization process, especially when C4 alkanes have first been removed.

As used herein, “binder” refers to the material used in the catalyst to provide necessary mechanical strength and/or resistance towards attrition loss. Common binders include clays, kaolin, attapulgite, boehmite, aluminas, silicas or combinations thereof. Binders are added in quantities higher than 20% in weight to reach the mechanical strength needed and form a homogeneous and plastic mixture. Binders used herein include, but are not limited to, silica, silica-alumina, bentonite, kaolin, bentonite with alumina, montmorillonite, attapulgite, titania, zirconia, and combinations thereof

As used herein, “silica” refers to SiO₂, “alumina” refers to Al₂O₃, “attapulgite” refers to a magnesium aluminum phyllosilicate, “titania” refers to titanium dioxide, and “zirconia” refers to zirconium dioxide.

Use of the term “optionally” with respect to any element of a claim means that the element is required, or alternatively, not required, both alternatives being within the scope of the claim.

Numbers and ranges disclosed herein may vary by some amount. Whenever a numerical range with a lower limit and an upper limit is disclosed, any number and any included range falling within the range is specifically disclosed. In particular, each range of values (of the form, “from about a to about b,” or, equivalently, “from approximately a to b,” or, equivalently, “from approximately a-b”) disclosed herein is to be understood to set forth each number and range encompassed within the broader range of values.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially” of excludes additional material elements, but allows the inclusions of non-material elements that do not substantially change the nature of the invention.

The terms in the claims have their plain, ordinary meaning unless otherwise explicitly and unambiguously defined by the patentee. Moreover, the indefinite articles “a” or “an”, as used in the claims, are defined herein to mean one or more than one of the elements that it introduces. If there is any conflict in the usages of a word or term in this specification and one or more patent or other documents, the definitions that are consistent with this specification should be adopted.

The following abbreviations are used herein:

| ABBREVIATION | TERM |
|---|---|
| B1 | 1-butene |
| B2 | 2-butene |
| EFF | effluent |
| FD | hydrocarbon feed |
| H-FER | Hydrogen ferrierite |
| IB1 | Isobutylene |
| PO/TBA | propylene oxide/t-butyl alcohol |
| WHSV | Weight hour space velocity (mass feed |
| | rate per hour per mass of catalyst |
| wt. % | weight percent |
| vol. % | volume percent |

### BRIEF DESCRIPTION OF THE DRAWINGS

FIG. 1A. Comparison of the conversion rate of isobutylene to normal butene of one embodiment of the present disclosure with methods of skeletal isomerization using an undiluted and diluted hydrocarbon feed, all performed at the same total hydrocarbon WHSV.

FIG. 1B. Yield of isobutylene of one embodiment of the present disclosure and a known method of skeletal isomerization.

FIG. 1C. Yield of C5+ heavies of one embodiment of the present disclosure and a known method of skeletal isomerization.

FIG. 2A. Comparison of the conversion rate of isobutylene to normal butene of one embodiment of the present disclosure and a known method of skeletal isomerization using a diluted hydrocarbon feed at a constant hydrocarbon feed:diluent ratio of 1:0.07.

FIG. 2B. Yield of isobutylene of one embodiment of the present disclosure and a known method of skeletal isomerization at a constant hydrocarbon feed:diluent ratio of 1:0.07.

FIG. 2C. Yield of C5+ heavies of one embodiment of the present disclosure and a known method of skeletal isomerization at a constant hydrocarbon feed:diluent ratio of 1:0.07.

FIG. 3A. Comparison of the conversion rate of isobutylene to normal butene of one embodiment of the present disclosure and a known method of skeletal isomerization using Catalyst 2.

FIG. 3B. Yield of isobutylene of one embodiment of the present disclosure and a known method of skeletal isomerization using Catalyst 2.

FIG. 3C. Yield of C5+ heavies of one embodiment of the present disclosure and a known method of skeletal isomerization using Catalyst 2.

FIG. 4. Length of the catalyst cycle for the isomerization of a diluted and undiluted hydrocarbon feed at various WHSV (g isobutylene/g catalyst/h).

### DETAILED DESCRIPTION

The disclosure provides a skeletal isomerization method for isomerizing olefins using a zeolite catalyst and an added hydrogen diluent feed to increase the lifetime of the catalyst before regeneration is needed. In some embodiments of the presently disclose method, a reduction in the formation of the heavy C5+ diolefins occur while increasing the formation of the skeletal isomer products. This results in an increase in the yield of isomerization products.

Conventional skeletal isomerization processes, both forward isomerization of linear olefins to branch olefins and reverse isomerization of branched olefins to linear olefins, employ catalysts, such as zeolites. These zeolite catalysts can be used with or without a refractory oxide binder material such as silica or alumina, and many are commercially available. However, these zeolite catalysts are susceptible to quick coking and subsequent blocking of pores, which lead to low cycle times before the catalyst must be de-coked and regenerated.

The presently disclosed methods overcome the issue of low cycle times by co-feeding a hydrogen diluent with the hydrocarbon feed. While diluents have been used to increase catalyst lifetimes, these diluents are typically inert gases such as helium, nitrogen, argon, or saturated hydrocarbons such as methane or n-butane. In the present methods, added hydrogen was unexpectedly found to increase the catalyst cycle beyond that of the inert gases, for both the forward and reverse isomerization process.

In some embodiments, the life cycle of the catalyst is at least 50% longer when hydrogen is used compared to processes that do not use a diluent, and at least 40% longer compared to processes that use an inert gas diluent. Under certain conditions, the hydrogen surprisingly extended the catalyst lifetime by 200%, compared to about 37% with helium as a diluent. Alternatively, the life cycle of the catalyst is extended by at least 3 days, 4 days, 5 days, 6 days, 7 days, or 8 days, when using hydrogen as a diluent compared to an inert gas diluent, when the WHSV is at least 2h⁻¹. Thus, under certain conditions, the catalyst cycle can be extended to at least sixteen days, at least 21 days, or at least 25 days in length.

The yield of skeletal isomer products by using the hydrogen diluent feed of this disclosure is increased due to the longer life cycle. In some embodiments, the yield of skeletal isomer products by using the hydrogen diluent feed of this disclosure can be 5 to 20% higher than using an inert gas diluent or no diluent. In some embodiment, the yield of the skeletal isomer products using the catalyst of this disclosure is at least 10% larger than using an inert gas diluent.

In some embodiments, the novel method presently disclosed comprises the steps of co-feeding a hydrocarbon feed that has at least one olefin at a hydrocarbon weight hour space velocity (WHSV) in the range of from 1 to 3011¹ and a hydrogen diluent feed into a reactor having an isomerization zeolite catalyst, wherein the reactor is maintained at a first temperature and a first pressure, and collecting one or more skeletal isomer olefin product. The at least one olefin in the feed can have two to ten carbons, and, during the co-feeding steps, a portion of the at least one olefin is isomerized into the at least one skeletal isomer olefin product. For example, if the at least one olefin is an iso-olefin such as isobutylene, then the skeletal isomer olefin product will be a linear olefin such as 1- or 2-butene. If the at least one olefin is a linear olefin such as 2-butene, then the skeletal isomer olefin product will be an iso-olefin such as isobutylene. The molar ratio of the hydrocarbon feed to the hydrogen diluent feed is in the range of about 1:0.01 to about 1:1.

In some embodiments, the novel method presently disclosed comprises the steps of co-feeding a hydrocarbon feed that has at least one olefin at a hydrocarbon weight hour space velocity (WHSV) in the range of from 1 to 3011¹ and a hydrogen diluent feed into a reactor having an isomerization zeolite catalyst, wherein the reactor is maintained at a temperature between 340° C. and 500° C. and a pressure between zero to about 1034 kPa (150 psig), and collecting one or more skeletal isomer olefin product. The at least one olefin in the feed can have two to ten carbons, and, during the co-feeding steps, a portion of the at least one olefin is isomerized into the at least one skeletal isomer olefin product. The molar ratio of the hydrocarbon feed to the hydrogen diluent feed is in the range of about 1:0.01 to about 1:1.

In some embodiments, the novel method presently disclosed comprises the steps of co-feeding a hydrocarbon feed that has at least one olefin at a hydrocarbon weight hour space velocity (WHSV) in the range of from 1 to 30⁻¹, and a hydrogen diluent feed into a reactor having an isomerization zeolite catalyst, wherein the reactor is maintained at a first temperature and a first pressure, and collecting one or more skeletal isomer olefin product, wherein the catalyst cycle is at least 40% longer than a method that does not use hydrogen as a diluent. The at least one olefin in the feed can have two to ten carbons, and, during the co-feeding steps, a portion of the at least one olefin is isomerized into the at least one skeletal isomer olefin product. The molar ratio of the hydrocarbon feed to the hydrogen diluent feed is in the range of about 1:0.01 to about 1:1.

More details on the skeletal isomerization process conditions and feeds are provided below.

Hydrocarbon Feedstream: The presently described methods are for the skeletal isomerization (both forward and reverse) of olefins, also known as alkenes. Thus, the hydrocarbon feedstream, or feed, used herein may comprises at least one olefin that will be isomerized into a skeletal isomer thereof. For example, an iso-olefin is a skeletal isomer of a linear olefin, and vice versa. In some embodiments, the at least one olefin in the hydrocarbon feed has two to ten carbon atoms.

In some embodiments, the hydrocarbon feed comprises unbranched linear, or normal olefins having two to ten carbons, as well as other hydrocarbons such as alkanes, di-olefins, aromatics, hydrogen, and inert gases. In other embodiments, the feed comprises at least 40 wt. % of linear C4 olefins, as well as other hydrocarbons such as alkanes, other olefins, and aromatics, and incidental gases (<5 vol. %) such as hydrogen and inert gases. Alternatively, the feed comprises at least 55 wt. % of linear C4 olefins, at least 70 wt. % of linear C4 olefins, at least 85 wt. % of linear C4 olefins, at least 95 wt. % of linear C4 olefins, or at least 99 wt. % of linear C4 olefins.

In other embodiments, the hydrocarbon feed used herein comprises branched olefins, also known as “iso-olefins”. In this disclosure, the branched olefins can have four to ten carbon atoms. In some embodiments, the feed used herein comprises a methyl-branched iso-olefin. In some embodiments of the disclosure, the feed contains isobutylene. As before, the hydrocarbon feed used in some embodiments of the disclosure may also include other hydrocarbons such as alkanes, di-olefins, and aromatics, as well as hydrogen and other gases.

In some embodiments of the disclosure, the feed comprises at least 40 wt. % isobutylene, at least 55 wt. % isobutylene, at least 70 wt. % isobutylene, at least 85 wt. % isobutylene, at least 95 wt. % isobutylene, or at least 99 wt. % isobutylene. The isobutylene can be from any source. In some embodiments, the isobutylene comes from a Raffinate 1 stream derived from a cracker/fluid catalytic cracking unit and has had the C4 alkanes removed. Alternatively, the isobutylene can come from a stream derived from a propylene oxide/t-butyl alcohol (PO/TBA) plant. The dehydration of the t-butyl alcohol can result in a more purified isobutylene stream than a stream sourced from a cracker.

Hydrogen Feed: The presently described methods co-feeds a hydrogen feedstream, or feed, alongside the hydrocarbon feed into the reactor. The added hydrogen feedstream is at least 70 vol. % of hydrogen and cannot contain any catalyst or reaction poisons. In some embodiments, the hydrogen feedstream has a high purity (e.g. 99.9998%). In other embodiments, the added hydrogen feedstream is a recycle stream that has at least 70 vol. % of hydrogen.

The amount of hydrogen feed utilized in the present methods can vary. In some embodiments, the molar ratio of hydrocarbon feed to hydrogen feed can be between 1:0.01 to 1:1; alternatively, the molar ratio of hydrocarbon feed to hydrogen feed is between 1:0.01 to 1:0.07; alternatively, the molar ratio of hydrocarbon feed to hydrogen feed is between 1:0.04 to 1:1; alternatively, the molar ratio of hydrocarbon feed to hydrogen feed is about 1:0.05 or 1:0.07. In other embodiments, the molar ratio of at least one olefin in the hydrocarbon feed to hydrogen feed can be between 1:0.01 to 1:1; alternatively, the molar ratio of at least one olefin in the hydrocarbon feed to hydrogen feed is between 1:0.01 to 1:0.07; alternatively, the molar ratio of at least one olefin in the hydrocarbon feed to hydrogen feed is between 1:0.04 to 1:1; alternatively, the molar ratio of at least one olefin in the hydrocarbon feed to hydrogen feed is about 1:0.05 or 1:0.07.

In other embodiments, the ratio of hydrocarbon feed to hydrogen feed can be between 2.5 vol. % and up to 50 vol. % of the total feed (both hydrocarbon and hydrogen) entering the reactor; alternatively, the ratio of hydrocarbon feed to hydrogen feed can be between 2.5 vol. % and up to 30 vol. % of the total feed (both hydrocarbon and hydrogen) entering the reactor; alternatively, the ratio of hydrocarbon feed to hydrogen feed can be between 25 vol. % and up to 50 vol. % of the total feed (both hydrocarbon and hydrogen) entering the reactor. The volume ratio can also be determined using the ratio of at least one olefin in the hydrocarbon feed to hydrogen feed, and will have the same percentage as described for the hydrocarbon feed, e.g. the ratio of at least one olefin in the hydrocarbon feed to hydrogen feed can be between 2.5 vol. % and up to 50 vol. % of the total volume of the at least one olefin and hydrogen entering the reactor

While larger amounts of hydrogen will also extend the catalyst lifetime, lower amounts (less than 50% of the total feed entering the reactor) are preferred from an economic perspective, depending on a user's ability to purge or reuse the spent hydrogen. For example, the isomerization products may need to be condensed after exiting the reactors. Depending on the isomerization products, it may be difficult to achieve a good separation of the hydrogen from the isomerization products without losing some of the isomerization products during the process. If higher amounts of hydrogen are used during the co-feeding steps, then higher amounts of isomerization products will be lost during the subsequent separation step.

Isomerization Catalyst: The isomerization catalyst used in embodiments of this disclosure includes catalysts suitable to skeletally isomerize olefins. This includes isomerizing iso-olefins to linear, or normal, olefins (unbranched) and vice versa.

In some embodiments of the disclosure, the catalyst may comprise a zeolite and such catalysts may be referred to as a “zeolite catalyst”. A zeolite catalyst used in embodiments of this disclosure may comprise a zeolite having one-dimensional channels with a channel diameter ranging from greater than about 0.42 nm to less than about 0.7 nm. Such zeolite catalysts may comprise zeolites channels with the specified diameter in one dimension. Zeolites having channel diameters greater than 0.7 nm are more susceptible to unwanted aromatization, oligomerization, alkylation, coking and by-product formation. However, under certain conditions, the coking may be beneficial, such as reducing the quantity of possible sites for the unwanted aromatization, oligomerization, alkylation.

Alternatively, the zeolite catalyst used in embodiments of this disclosure may comprise two or three-dimensional zeolites having a channel size greater than 0.34 nm in two or more dimensions permit dimerization and trimerization of the alkene. Hence, zeolites having a channel diameter bigger than about 0.7 nm in any dimension or having a two or three-dimensional channel structure in which any two of the dimensions has a channel size greater than about 0.42 nm, while not suitable for isomerization of isobutylene, may nevertheless be used in light of the preferential coking conditions described in the present disclosure. Examples of zeolites that can be used in the processes of this disclosure include the hydrogen form of ferrierite (H-FER), the hydrogen form of heulandite, the hydrogen form of stilbite, SAPO-11, SAPO-31, SAPO-41, Z SM-12, ZSM-22, ZSM-23, ZSM-35, and ZSM-48. The isotypic structures of these frameworks, known under other names, are considered to be equivalent.

In some embodiments of the present disclosure, the zeolite catalyst is H-ferrierite (H-FER). H-FER is derived from ferrierite, a naturally occurring zeolite mineral having a composition varying somewhat with the particular source. A typical elemental composition of ferrierite is described as:

Na₂Mg₂[Al₆Si₃₀O₇₂].18H₂O.

The prominent structural features of ferrierite found by x-ray crystallography are perpendicular channels in the alumino-silicate framework—one with 8-membered rings in the [010] direction and one with 10-membered rings in the [001] direction. These channels, which are roughly elliptical in cross-section, are of two sizes: larger channels having major and minor axes of 0.54 and 0.42 nm, respectively, and smaller parallel channels having major and minor axes of 0.48 and 0.35 nm, respectively. Conversion of ferrierite to its hydrogen form, H-ferrierite, replaces sodium cations with hydrogen ions in the crystal structure, making it more acidic. Both the alkali metal and hydrogen forms reject multiple branched chain and cyclic hydrocarbon molecules and retard coke formation.

The zeolite catalyst used in the presently disclosed methods may also have a silica to alumina ratio (SAR) of about 10:1 to about 100:1. In some embodiments, the SAR of the catalyst used in the presently described methods is about 20, about 40, about 60 or about 80.

Further, the zeolite catalyst used in the presently disclosed methods may contain hydrogenation-active components such as palladium.

The zeolite catalyst used in embodiments of the present disclosure may be used alone or suitable combined with a refractory oxide that serves as a binder material. Suitable refractory oxides include, but are not limited to, natural clays, such as bentonite, montmorillonite, attapulgite, and kaolin; alumina; silica; silica-alumina; hydrated alumina; titania; zirconia and mixtures thereof. The weight ratio of binder material and zeolite suitably ranges from 1:10 to 10:1. In some embodiments of the disclosure, the weight ratio of binder to zeolite is in the range of 1:10 to 5:1, the range of 3:5 to 10:1, or the range of 3:5 to 8:5.

The catalyst in some embodiments of the presently disclosed methods, when combined with at least one binder, can be extruded into any shape. This includes, but is not limited to, spheres, pellets, tablets, platelets, cylinders, helical lobed extrudate, trilobes, quadralobes, multilobed (5 or more lobes), and combinations thereof.

In some embodiments, the catalyst is a pure zeolite powder. In other embodiments, the catalyst is a bound zeolite that has been extruded in a trilobed, quadralobe, or multilobed shape. In yet other embodiments, the catalyst is a pure H-FER powder. In some embodiments, the catalyst is a pure H-FER powder with a SAR of 80. Alternatively, the catalyst is an H-FER that is bound and extruded in a trilobed, quadralobe, or multilobed shape. In yet another alternative, the catalyst is an H-FER that is bound and has a SAR of 80.

Operating Conditions for Skeletal Isomerization Process: In embodiments of the disclosure, the hydrocarbon feed and hydrogen feed may be contacted with the isomerization catalyst under reaction conditions effective to skeletally isomerize the olefins therein. This contacting step may be conducted in the vapor phase by bringing a vaporized hydrocarbon and hydrogen feed into contact with the solid isomerization catalyst. The hydrocarbon feed, hydrogen feed, and/or catalyst can be preheated as desired.

The isomerization process of the disclosure may be carried out in a variety of reactor types. In some embodiments of the disclosure, the reactor is a packed bed reactor. In some embodiments of the disclosure, the reactor is a fixed bed reactor. In some embodiments of the disclosure, the reactor is a fluidized bed reactor. In some embodiments of the disclosure, the reactor is a moving bed reactor. In embodiments of the disclosure using a moving bed reactor, the catalyst bed may move upwards or downwards.

The temperature of the reactor can vary from about 250° C. to about 600° C., or from about 380° C. to about 425° C. Alternatively, the reactor temperature for the isomerization is between about 250° C. to about 420° C., about 400 and 600° C., or about 340° and 500° C. In yet another alternative, the reactor temperature is about 418° C.

The reaction pressure conditions can vary from about zero to about 1034 kPa (150 psig), or from about zero to about 345 kPa (50 psig). Alternatively, the reaction pressure for the isomerization is between about 34 kPa (5 psig) to about 345 kPa (50 psig), about 34 kPa (5 psig) to about 83 kPa (12 psig), 55 kPa (8 psig) to about 138 kPa (20 psig), or 55 kPa (8 psig) to about 97 kPa (14 psig). In yet another alternative, the pressure is about 69 kPa (10 psig).

The weight hourly space velocity (WHSV) feed rates of the hydrocarbon feed can range from about 1 to about 200 hⁱ, with the hydrogen diluent. In some embodiments, the weight hourly space velocity feed rates are from about 1 to about 30 h⁻¹; alternatively, the weight hourly space velocity feed rates are from about 1 to about 10 h⁻¹; alternatively, the weight hourly space velocity feed rates are from about 2 to about 7 h⁻¹; alternatively, the weight hourly space velocity feed rate is about 2 to about 4

The higher the WHSV feed rates, the shorter the life cycle of the catalyst. By performing a skeletal isomerization using the steps above, the life cycle of the catalyst increases even at higher WHSV feed rates compared to an isomerization process that does not use hydrogen as a diluent. The life cycle of the catalyst can be extended by at least 3 days, 4 days, 5 days, 6 days, 7 days, or 8 days, when using hydrogen as a diluent compared to an inert gas diluent, when the WHSV is at least 2 h¹. Similar extensions in the life cycle of the catalyst are observed when the WHSV is much faster.

In some embodiments, the ratio of time on stream for the olefin conversion to reach 45% to linear butene (nB) yield is greater than 5.5:1 at an olefin feed weight hourly space velocity (WHSV) of 2 (g olefin/g catalyst/hr). In some embodiments, the ratio of time on stream for the olefin conversion to reach 45% to linear butene (nB) yield is greater than 5.75:1 at an olefin feed weight hourly space velocity (WHSV) of 2 (g olefin/g catalyst/hr). In some embodiments, the ratio of time on stream for the olefin conversion to reach 45% to linear butene (nB) yield is greater than 6.0:1 at an olefin feed weight hourly space velocity (WHSV) of 2 (g olefin/g catalyst/hr). In some embodiments, the ratio of time on stream for the olefin conversion to reach 45% to linear butene (nB) yield is greater than 6.25:1 at an olefin feed weight hourly space velocity (WHSV) of 2 (g olefin/g catalyst/hr).

In some embodiments, the ratio of time on stream for the olefin conversion to reach 45% to linear butene (nB) yield is greater than 4.5:1 at an olefin feed weight hourly space velocity of 3 (g olefin/g catalyst/hr). In some embodiments, the ratio of time on stream for the olefin conversion to reach 45% to linear butene (nB) yield is greater than 4.75:1 at an olefin feed weight hourly space velocity of 3 (g olefin/g catalyst/hr). In some embodiments, the ratio of time on stream for the olefin conversion to reach 45% to linear butene (nB) yield is greater than 5.0:1 at an olefin feed weight hourly space velocity of 3 (g olefin/g catalyst/hr). In some embodiments, the ratio of time on stream for the olefin conversion to reach 45% to linear butene (nB) yield is greater than 5.25:1 at an olefin feed weight hourly space velocity of 3 (g olefin/g catalyst/hr).

In some embodiments, the ratio of time on stream for the olefin conversion to reach 45% to linear butene (nB) yield is greater than 2.75:1 at an olefin feed weight hourly space velocity of 5 (g olefin/g catalyst/hr). In some embodiments, the ratio of time on stream for the olefin conversion to reach 45% to linear butene (nB) yield is greater than 2.90:1 at an olefin feed weight hourly space velocity of 5 (g olefin/g catalyst/hr). In some embodiments, the ratio of time on stream for the olefin conversion to reach 45% to linear butene (nB) yield is greater than 3.0:1 at an olefin feed weight hourly space velocity of 5 (g olefin/g catalyst/hr). In some embodiments, the ratio of time on stream for the olefin conversion to reach 45% to linear butene (nB) yield is greater than 3.15:1 at an olefin feed weight hourly space velocity of 5 (g olefin/g catalyst/hr).

In some embodiments, the ratio of time on stream for the olefin conversion to reach 45% to linear butene (nB) yield is greater than 3.0:1 at an olefin feed weight hourly space velocity of 7 (g olefin/g catalyst/hr). In some embodiments, the ratio of time on stream for the olefin conversion to reach 45% to linear butene (nB) yield is greater than 3.25:1 at an olefin feed weight hourly space velocity of 7 (g olefin/g catalyst/hr). In some embodiments, the ratio of time on stream for the olefin conversion to reach 45% to linear butene (nB) yield is greater than 3.40:1 at an olefin feed weight hourly space velocity of 7 (g olefin/g catalyst/hr). In some embodiments, the ratio of time on stream for the olefin conversion to reach 45% to linear butene (nB) yield is greater than 3.55:1 at an olefin feed weight hourly space velocity of 7 (g olefin/g catalyst/hr).

By performing a skeletal isomerization using the steps above, the yield of the skeletal isomer product also increases compared to an isomerization process that does not use hydrogen as a diluent. The yield of skeletal isomerization products by using the hydrogen diluent feed of this disclosure can be 5 to 20% higher than using an inert gas diluent or no diluent.

Using the above-described methods, the skeletal isomerization process is improved because the catalyst cycle is longer, allowing for a greater amount of structurally isomerized product, also called skeletal isomer olefin product, to be formed. In some embodiments, when the feed comprises C4 olefins, a greater amount of the desired structurally isomerized product can be formed. This leads to a more cost-effective isomerization process for generating greater amounts of structurally isomerized C4 olefins.

### EXAMPLES

The following examples are included to demonstrate embodiments of the appended claims using the above-described methods for increasing the yield of structural isomerization products for an isobutylene feed. The example is intended to be illustrative, and not to unduly limit the scope of the appended claims. Those of skill in the art should appreciate that many changes can be made in the specific embodiments which are disclosed and still obtain a like or similar result without departing from the spirit and scope of the disclosure herein. In no way should the following examples be read to limit, or to define, the scope of the appended claims.

Hydrocarbon Feed. For Examples 1-3, the hydrocarbon feed comprised 99.95 wt. % of isobutylene. The skeletal isomer product olefins for such as feed composition include 1-butene, trans-2-butene, and cis-2-butene.

Hydrogen feed: For Examples 1-3, the hydrogen feed had a research grade purity (99.9999%).

Helium feed: For Examples 1-3, the helium feed was 99.9995% purity.

Calculations. For Examples 1-3 below, the conversion of reactants to products is calculated. Without being bound by theory, it is believed that during the isomerization reaction, equilibrium is achieved between, for example, the isobutylene, 1-butene and trans- and cis-2-butene. Therefore, for Examples 1-3 wherein isobutylene is the hydrocarbon feedstream, the calculation of conversion reflects the hydrocarbon feed (FD) and effluent (EFF) concentrations of 1-butene (B1), 2-butene (B2), and isobutylene (IB1). Conversion is calculated as:

$$\% isobutylene Conversion = \frac{( wt \% IB 1 ) FD - ( wt \% IB 1 ) EFF}{( wt \% IB 1 ) F D} \times 100$$

Yield is calculated as

$$\% linear butene Yield = \frac{( wt \% B 1 + wt \% B 2 ) EFF - ( wt \% B 1 + wt \% B 2 ) F D}{( wt \% IB 1 ) FD} \times 100$$

Development of equivalent equations for other olefin reactants and skeletal isomer products are well within the abilities of one with skill in the art. For instance, if the hydrocarbon feed was 1-butene, the following equation can be used for the determination of the conversion of linear C4 olefins to isobutylene:

$$\% linear C 4 butene Conversion = 1 - \frac{( wt \% B 1 ) FD - ( wt \% B 1 + wt \% B 2 ) EFF}{( wt \% B 1 ) FD} \times 100$$

### Example 1

Isomerization of isobutylene was performed in Example 1 using the method of this disclosure, and compared to Comparative Example 1 that uses helium as a diluent and Comparative Example 2 that does not have a diluent feed.

In accordance with the presently described methods, the method in Example 1 comprised co-feeding 99.95 wt. % of isobutylene and the hydrogen feed through a fixed bed reactor at approximately 418° C. The fixed bed reactor contained Catalyst 1, a commercially available bound hydrogen ferrierite (H-FER) catalyst with a SAR of 80, and palladium as one component in the catalyst. No catalyst pretreatment was performed other than heating to reaction temperature under an inert gas, helium. Once the reaction temperature was reached, the feed(s) were introduced. The isobutylene feed was maintained at WHSV=2 (2 g isobutylene/g catalyst/h), and the hydrogen feed speed was adjusted to maintain specific molar ratios of isobutylene to diluent. Example 1 began with a molar ratio of 1:0.5; however, the molar ratio was increased to 1:1 at about the 75 hour mark, decreased to 1:0.5 at about 120 hours and 1:0 at about 170 hours, before being increased back to 1:0.5. The different molar ratios were used to determine the effect on product distribution.

Comparative Example 1 was performed with a helium feed as the diluent. The hydrocarbon feed, catalyst, reactors, and molar ratio schemes are the same as above. Comparative Example 2 was performed without a diluent feed. The hydrocarbon feed, catalyst, and reactors in Comparative Example 2 are the same as above.

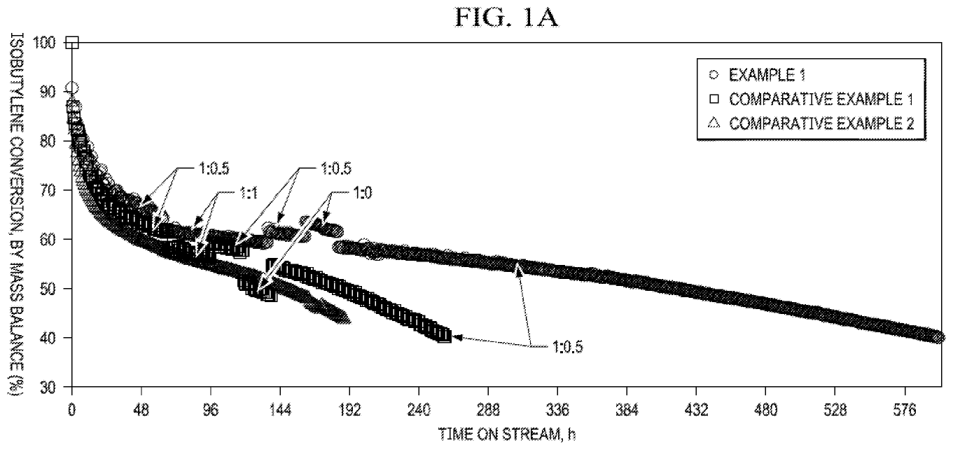

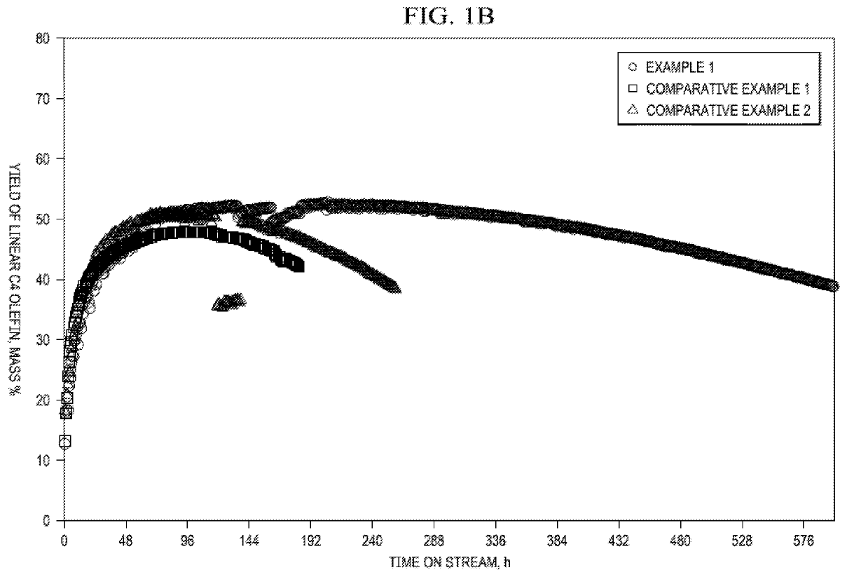

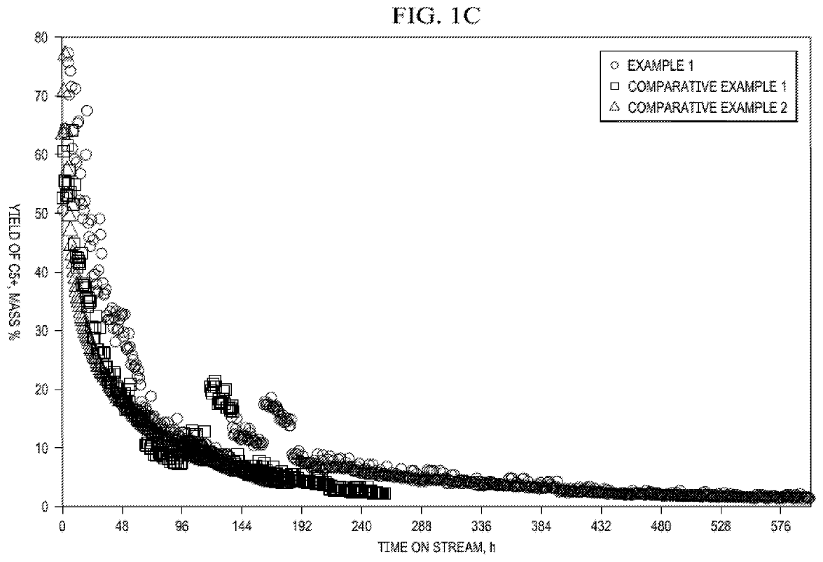

The results for Example 1, and Comparative Examples 1 and 2 are displayed in FIGS. 1A-1C and Table 1. Data collection stopped when a conversion rate of about 40 was reached.

The conversion rate of isobutylene to linear butenes and the catalyst cycle are displayed in FIG. 1A. The isobutylene conversion for Example 1 is much longer than either comparative example. Helium extended the catalyst cycle to 11 days, which is about 3 days longer than the 8 day catalyst cycle when no diluent was used. However, using hydrogen as a diluent more than doubled the extension of time, from about 8 days with no diluent to about 16 days with hydrogen. The doubling of the catalyst life cycle translates into cost saving in both the amount of catalyst and the fewer interruption on operation.

Further, the amount of isobutylene conversion was much higher for Example 1. As shown in Table 1, the amount of time it took to reach 45% conversion of isobutylene in Example 1 was more than double that observed in Comparative Examples 1 and 2.

The yield of reaction products is shown in FIGS. 1B and 1C. The yield of linear butenes in the reaction for Example 1 is much higher than that in the Comparative Example 1 and 2, as shown in FIG. 1B. The addition of diluents causes a closer-to-equilibrium yield of linear butene and extends the time over which linear butene are formed during the cycle. Table 1 displays a snapshot of the cumulative yield (mass based) of products at a 45% conversion rate for each example. In addition to the extended cycle length, the yield of skeletal isomer products, here linear butene (nB), is improved by at least 7% with the addition of hydrogen.

TABLE 1
Cumulative product distribution at 45% conversion rate

| | Isobutylene | Light olefins | | | Ratio |
|---|---|---|---|---|---|
| | conversion, | (C1-C3) and | | | (IB_{conv.}/ |
| | h | Saturates | C5+ | nB | nB_{yield}) |
| Example 1 | 512.7 | 1.85 | 9.78 | 46.70 | 10.98 |
| Comp. Ex. 1 | 223.8 | 2.62 | 13.27 | 43.41 | 5.16 |
| Comp. Ex. 2 | 182.8 | 2.28 | 13.88 | 42.34 | 4.32 |

nB = linear butene
Comp. Ex.—comparative example

The initial yield of undesired heavy C5+ olefin, as shown in FIG. 1C, is slightly elevated with the addition of the hydrogen diluent. However, the total amount of C5+ products over the entire cycle is ultimately less, as shown in Table 1. This trend is also seen with the light olefins and saturates. As more linear butene is produced, less by-products are produced.

### Example 2

The results in Example 1 show that adding a hydrogen diluent will increase the catalyst cycle, as compared to a similar process using other gases as a diluent, and subsequently increase the yield of linear butenes. Hydrogen will have to be separated from the isomerization products and some plants may require the use of a much smaller amount of hydrogen to reduce separation costs. Additionally, while there is potential to reuse the separated hydrogen in other on-site processes, some plants may prefer to keep hydrogen usages to a minimum. As such, the ability to increase the catalyst cycle was evaluated for a smaller molar ratio of diluents in this example to determine if the positive benefit of better yields and longer cycle length is realized.

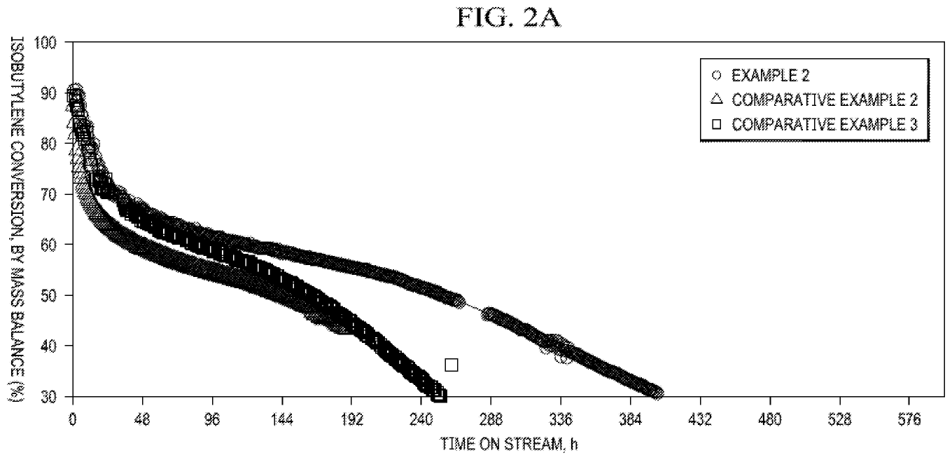

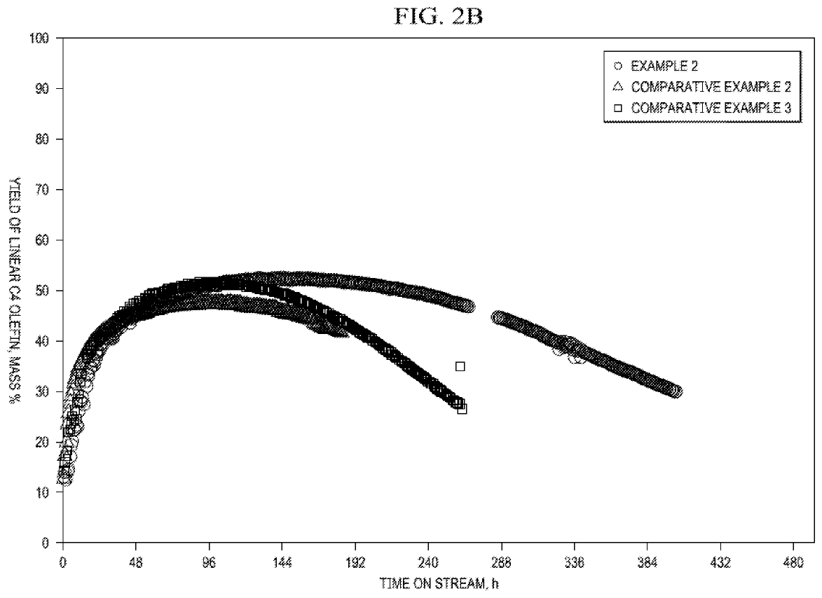

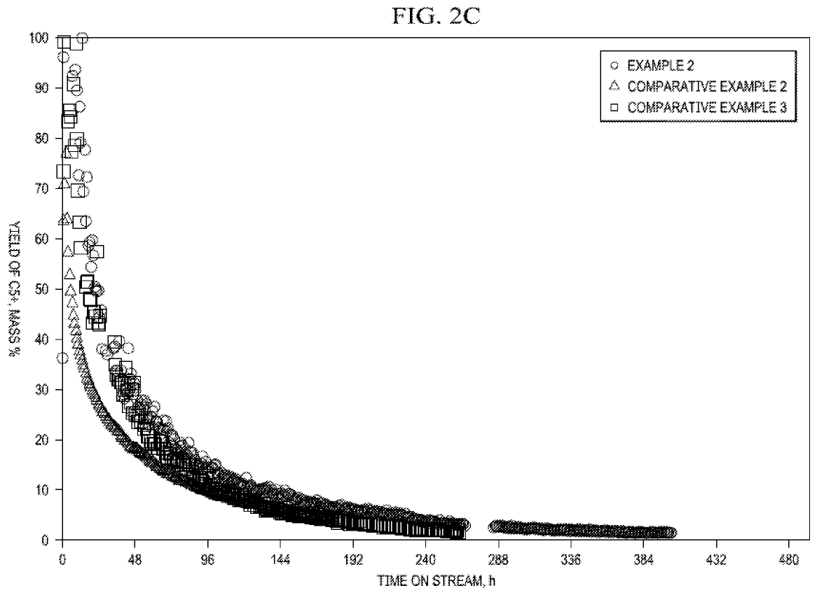

Example 2 used the same hydrocarbon feed, catalyst, and reactors as Example 1, except the molar ratio of hydrocarbon to hydrogen was held at 1 to 0.07. Comparative Example 3 was performed with a helium feed as the diluent, with a molar ratio of hydrocarbon to helium of 1 to 0.07. The same hydrocarbon feed, catalyst, and reactors are the same as above. The results are shown in FIGS. 2A-C and Table 2.

The conversion rate of isobutylene to linear butenes and the catalyst cycle are displayed in FIG. 2A. Comparative Example 2, without a diluent, reached a 45% conversion rate at about 182 h. The cycle length to this same conversion rate for Comparative Example 3 is not extended much. In fact, the addition of helium at a molar ratio of 1:0.07 isobutylene to helium reached 191 hours, which is less than an extra day compared to Comparative Example 2. However, the addition of 1:0.07 molar ratio isobutylene to hydrogen in Example 2 extends the cycle length time to reach a 45% conversion rate to 298 hours. This is an extension of about 6 days—well above the extension seen with the helium in Comparative Example 3.

The yield of reaction products is shown in FIGS. 2B and 2C. The yield of linear butenes in the reaction for Example 2 is much higher than that in the Comparative Example 2 and 3, as shown in FIG. 2B. The addition of diluents causes a closer-to-equilibrium yield of linear butene and extends the time over which linear butene are formed during the cycle.

Table 2 displays a snapshot of the cumulative yield (mass based) of products at a 45% conversion rate for each example. In addition to the extended cycle length, the yield of linear butene (nB) is improved by at least 3.7% with the addition of hydrogen. The decrease in yield is attributed to the smaller amount of hydrogen being used in this example. However, it should be noted that less heavy C5+ olefins were produced using hydrogen, compared to helium.

TABLE 2
Cumulative product distribution at 45%
conversion rate for FIG. 2B and 2C

| | Isobutylene | Light olefins | | | Ratio |
|---|---|---|---|---|---|
| | conversion, | (C1-C3) and | | | (IB_{conv.}/ |
| | h | Saturates | C5+ | nB | nB_{yield}) |
| Example 2 | 298 | 2.21 | 14.28 | 44.73 | 6.66 |
| Comp. Ex. 3 | 191 | 2.16 | 17.15 | 43.13 | 4.43 |
| Comp. Ex. 2 | 182.8 | 2.28 | 13.88 | 42.34 | 4.32 |

nB = linear butene
Comp. Ex.—comparative example

These results show that even small amounts of hydrogen can increase the catalyst cycle compared to other diluents or no diluents.

### Example 3

The effect of the isomerization catalyst was also evaluated. Some catalyst, such as that used in Examples 1 and 2 contain hydrogenation-active components such as palladium. In this example, an isomerization catalyst without a hydrogenation-active component was used to determine if the improved catalyst cycle experienced with a hydrogen diluent was catalyst specific.

In accordance with the presently described methods, the method in Example 3 comprised co-feeding 99.95 wt. % of isobutylene and the hydrogen feed through a fixed bed reactor at approximately 418° C., same as Examples 1 and 2. In contrast to Examples 1 and 2, the fixed bed reactor contained Catalyst 2, a commercially available unbound H-FER catalyst powder with a SAR of 80, and no palladium or other hydrogenation-active components. No catalyst pretreatment was performed other than heating to reaction temperature under an inert gas as described in Example 1. The isobutylene feed was maintained at WHSV=2 (2 g isobutylene/g catalyst/h), and the hydrogen feed speed was adjusted to maintain specific molar ratio of isobutylene to hydrogen of 1:0.5.

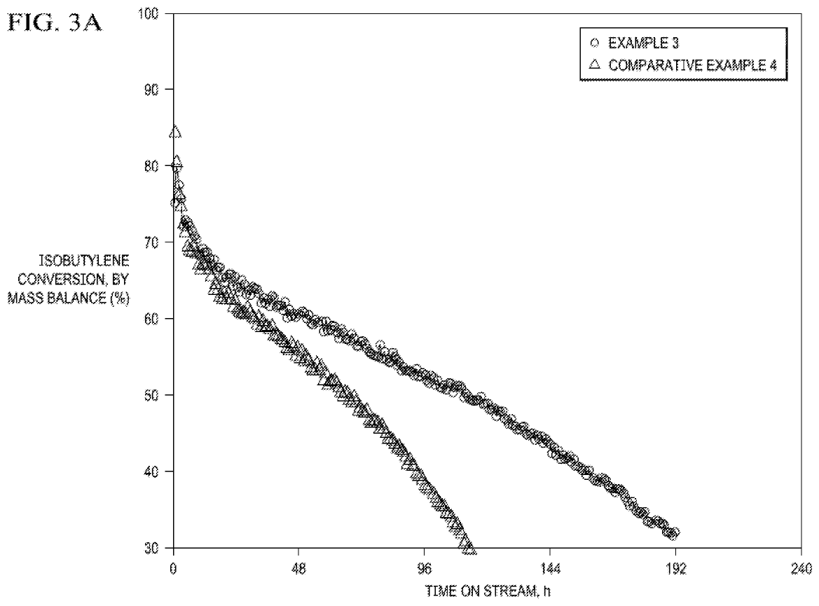

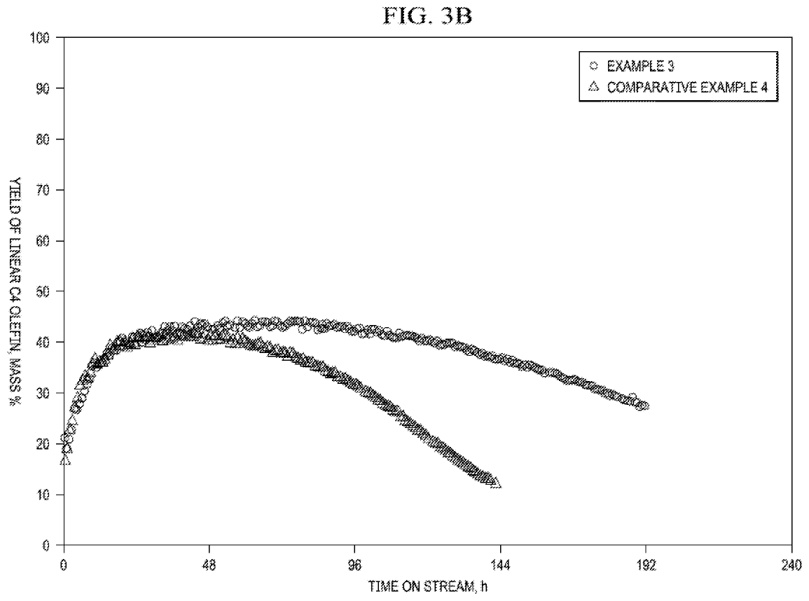

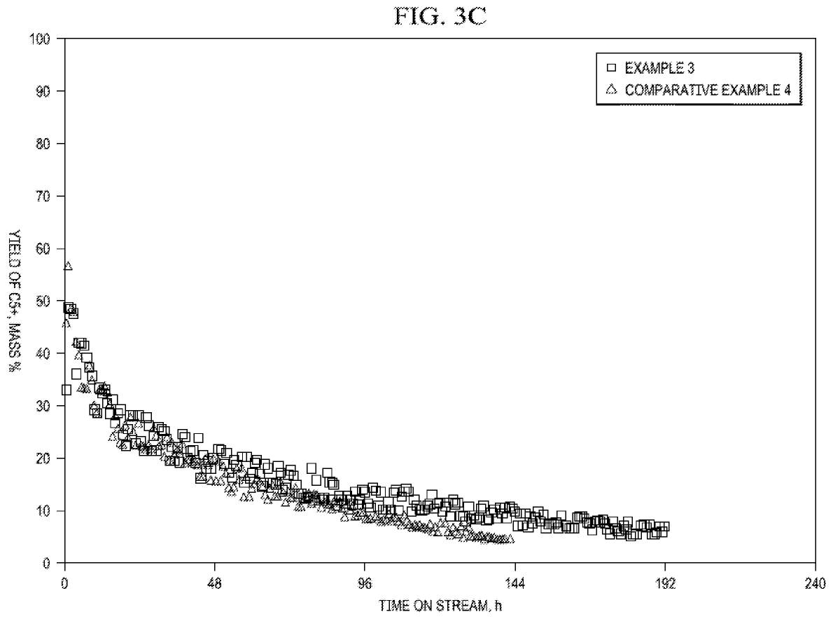

Comparative Example 4 was performed with a helium feed as the diluent, with a molar ratio of hydrocarbon to helium of 1 to 0.5. The hydrocarbon feed, catalyst, and reactors are the same as Example 3. The results are shown in FIGs. 3A-3C and Table 3.

The conversion rate of isobutylene to linear butenes and the catalyst cycle are displayed in FIG. 3A. The cycle length observed for Comparative Example 4 is much shorter than that of Example 3. Using the 45% conversion rate as a set point, the cycle length was extended by 66% using hydrogen, resulting in an increase of more than two days.

The yield of reaction products is shown in FIGS. 3B and 3C. Similar to Examples 1 and 2, the yield of linear butenes in the reaction for Example 3 is much higher than that in the Comparative Example 4, as shown in FIG. 3B. The addition of diluents causes a closer-to-equilibrium yield of linear butene and extends the time over which linear butene are formed during the cycle. This is further supported by Table 3. In addition to the extended cycle length, the yield of linear butene (nB) is improved by at least 6% with the addition of hydrogen, and less heavy C5+ olefins were produced using hydrogen.

TABLE 3
Cumulative product distribution at 45%
conversion rate for FIG. 3B and 3C

| | Isobutylene | Light olefins | | | Ratio |
|---|---|---|---|---|---|
| | conversion, | (C1-C3) and | | | (IB_{conv.}/ |
| | h | Saturates | C5+ | nB | nB_{yield}) |
| Example 3 | 136.4 | 4.32 | 17.21 | 38.31 | 3.56 |
| Comp. Ex. 4 | 82.1 | 4.72 | 19.77 | 36.01 | 2.28 |

nB = linear butene
Comp. Ex.—comparative example

These results show that hydrogen can increase the catalyst cycle compared to other diluents, regardless of the type of zeolite catalyst used. Thus, catalyst with hydrogenation-active components are not needed to achieve the observed increase in catalyst cycle length. However, as demonstrated in other Examples, the use of a catalyst having a hydrogenation-active component increases catalyst cycle length.

### Example 4

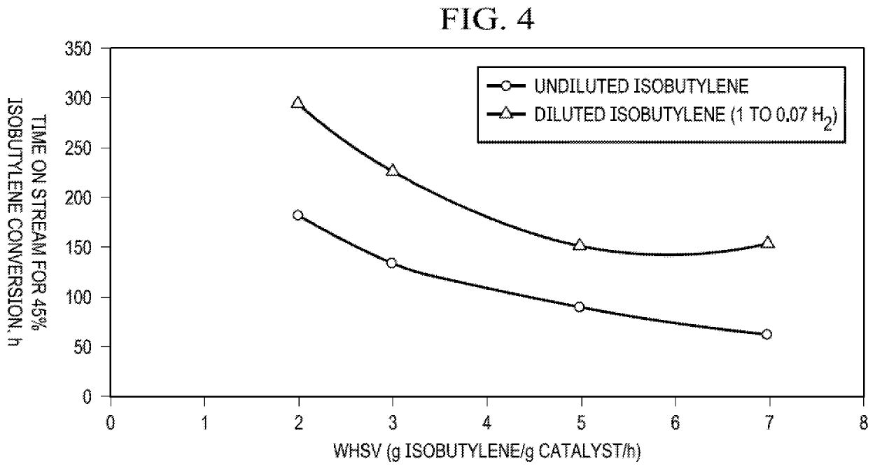

The effect of WHSV feed rates was also evaluated. Example 4 used the same hydrocarbon feed, catalyst and reactors as Example 1, except the isobutylene feed was maintained at WHSV=2, 3, 5, or 7 (g isobutylene/g catalyst/h) during each isomerization reaction. For the diluted isobutylene feed, the hydrogen feed speed was adjusted to maintain a constant hydrocarbon feed:diluent ratio of 1:0.07 for each reaction. The fixed bed reactor was at approximately 418° C. during each isomerization for this example. Isomerization reactions with undiluted isobutylene feeds (pure′ isobutylene feeds) without the hydrogen were also performed at various WHSV for comparison. The results for Example 4 are shown in FIG. 4 and Table 4.

The length of the catalyst cycle was longer at all WHSV feed rates when the hydrocarbon feed was diluted with hydrogen, compared to an undiluted hydrocarbon feed. As shown in FIG. 4, the life cycle of the catalyst was increased by at least about 40% at each feed rate. The highest increase in life cycle was seen when at the higher feed rates of WHSV=7. Here, the life cycle of the catalyst lasted for about 60% longer with the diluted hydrocarbon feed instead of the pure hydrocarbon feed.

TABLE 4
Cumulative product distribution at 45% conversion rate for FIG. 4

| | Isobutylene | Light olefins | | | Ratio |
|---|---|---|---|---|---|
| | conversion, | (C1-C3) and | | | (IB_{conv.}/ |
| WHSV | h | Saturates | C5+ | nB | nB_{yield}) |
| 2 (Example 2) | 298 | 2.21 | 14.28 | 44.73 | 6.66 |
| 2 (Comp. Ex. 2) | 182.8 | 2.28 | 13.88 | 42.34 | 4.32 |
| 3 (undiluted) | 135 | 2.28 | 19.46 | 37.43 | 3.61 |
| 3 (diluted) | 225 | 3.52 | 19.79 | 40.63 | 5.54 |
| 5 (undiluted) | 90 | 2.84 | 16.57 | 40.17 | 2.24 |
| 5 (diluted) | 156 | 3.11 | 11.67 | 46.04 | 3.39 |
| 7 (undiluted) | 62 | 2.94 | 24.04 | 33.69 | 1.84 |
| 7 (diluted) | 154 | 3.03 | 16.61 | 39.86 | 3.86 |

nB = linear butene
Comp. Ex.—comparative example

Further, the increases in yield of linear butenes were observed at the various WHSV feed rates. In addition to the extended life cycle, the yield of linear butene (nB) is improved by more than 8% with the addition of hydrogen as a diluent and at faster feed rates (>2). As mentioned before, the production of more linear butene means less byproducts are produced.

### Example 5

The isomerization products produced using hydrogen as a diluent were further characterized by evaluating the differences in the C5+ liquid product. The C5+ stream can be used for gasoline and low diolefin content leads to more favorable gasoline blending requirements. Aliquots of the liquid condensate remaining at the end of example were collected at ambient temperatures. A semi-quantitate individual species and diolefin analysis was performed using Electron Ionization (EI) Mass Spectrometry and NIST 14 Library. Differences in peak areas and identified dienes were investigated for relative changes in diolefinic content after hydrogen and helium co-fed hydrotreatment. The results are given in Table 5.

TABLE 5
Analysis of C5+ heavy condensate

| | % diolefin area |
|---|---|
| Example 1 | 3.6 |
| Comp. Ex. 1 | 10.5 |
| Example 2 | 4.8 |
| Comp. Ex. 3 | 6.3 |

Comp. Ex.—comparative example

The amount of diolefin produced during the hydrogen runs (Examples 1 and 2) were less than the comparative examples using helium. The largest difference in diolefin production was observed when more diluent was co-fed with the hydrocarbon feed. These results indicate that the addition of hydrogen alters the nature of the C5+ heavy condensate, and that the amount of diolefins in the liquid product is reduced when hydrogen is co-fed. Thus, these streams will have more favorable gasoline blending.

Examples 1 through 5 show that the use of hydrogen as a diluent not only increases the length of the catalyst cycle, even when small amounts of hydrogen or faster feed rates are utilized, but also increases the amount of skeletal isomer products, compared to processes that utilize inert gases as diluents.

### ADDITIONAL DISCLOSURE

The particular embodiments disclosed above are merely illustrative, as the present disclosure may be modified and practiced in different but equivalent manners apparent to those skilled in the art having the benefit of the teachings herein. Furthermore, no limitations are intended as to the details of construction or design herein shown, other than as described in the claims below. It is therefore evident that the particular illustrative embodiments disclosed above may be altered of modified and such variations are considered within the scope and spirit of the present disclosure. Alternative embodiments that result from combining, integrating, and/or omitting features of the embodiment(s) are also within the scope of the disclosure. While compositions and methods are described in broader terms of “having”, “comprising,” “containing,” or “including” various components or steps, the compositions and methods can also “consist essentially of or “consist of the various components and steps. Use of the term “optionally” with respect to any element of a claim means that the element is present, or alternatively, the element is not present, both alternatives being within the scope of the claim.

Numbers and ranges disclosed above may vary by some amount. Whenever a numerical range with a lower limit and an upper limit is disclosed, any number and any included range falling within the range is specifically disclosed. In particular, each range of values (of the form, “from about a to about b,” or, equivalently, “from approximately a to b,” or, equivalently, “from approximately a-b”) disclosed herein is to be understood to set forth each number and range encompassed within the broader range of values. Also, the terms in the claims have their plain, ordinary meaning unless otherwise explicitly and unambiguously defined by the patentee. Moreover, the indefinite articles “a” or “an”, as used in the claims, are defined herein to mean one or more than one of the element that it introduces. If there is any conflict in the usages of a word or term in this specification and one or more patent or other documents, the definitions that are consistent with this specification should be adopted.

Embodiments disclosed herein include:

A: A skeletal isomerization process comprising the steps of: co-feeding a hydrocarbon feed comprising at least one olefin and a hydrogen feed to a reactor containing an isomerization zeolite catalyst; and isomerizing the at least one olefin to at least one skeletal isomer product in the reactor for at least one catalyst cycle.

B: A skeletal isomerization process comprising the steps of: co-feeding a hydrocarbon feed comprising at least one olefin and a hydrogen feed to a reactor containing an isomerization zeolite catalyst, wherein the hydrocarbon feed is fed at a weight hourly space velocity (WHSV) between 1 to 30⁻¹; and isomerizing the at least one olefin to at least one skeletal isomer product in the reactor for at least one catalyst cycle, wherein the catalyst cycle is at least sixteen days.

C: A skeletal isomerization process comprising the steps of: co-feeding a hydrocarbon feed comprising at least one olefin and a hydrogen feed to a reactor containing an isomerization zeolite catalyst, wherein the hydrocarbon feed is fed at a weight hourly space velocity (WHSV) between 1 to 30 h⁻¹ and a molar ratio of the hydrocarbon feed to the hydrogen feed is between about 1:0.01 to about 1:1; and isomerizing the at least one olefin to at least one skeletal isomer product in the reactor for at least one catalyst cycle, wherein the catalyst cycle is at least sixteen days, a temperature of the reactor is from about 340° C. to about 500° C., and the isomerization zeolite catalyst is the hydrogen form of ferrierite (H-FER).

Each of embodiments A, B, and C may have one or more of the following additional elements:

Element 1: further comprising the step of recovering the at least one skeletal isomer product from the reactor. Element 2: wherein the hydrocarbon feed is fed at a weight hourly space velocity (WHSV) between 1 to 30h⁻¹. Element 3: wherein the isomerization zeolite catalyst is the hydrogen form of ferrierite (H-FER). Element 4: wherein a molar ratio of the at least one olefin in the hydrocarbon feed to the hydrogen feed during the co-feeding step is between about 1:0.01 to about 1:1. Element 5: wherein a molar ratio of the hydrocarbon feed to the hydrogen feed during the co-feeding step is between about 1:0.01 to about 1:1. Element 6: wherein the ratio of the hydrocarbon feed to the hydrogen feed is between about 2.5 vol. % and up to 50 vol. %, based on the volume of the total feed. Element 7: wherein the at least one olefin is an iso-olefin. Element 8: wherein the at least one olefin is isobutylene and the at least one skeletal isomer product is 1-butene and 2-butene. Element 9: wherein the at least one olefin comprises 1-butene and 2-butene, and the at least one skeletal isomer product is isobutylene. Element 10: wherein a temperature of the reactor is from about 340° C. to about 500° C. Element 11: wherein a pressure of the reactor is from zero to about 345 kPa (50 psig). Element 12: wherein the hydrocarbon feed comprises at least 40 wt. % isobutylene. Element 13: wherein the ratio of time on stream for the at least one olefin conversion to reach 45% to linear butene (nB) yield is: (i) greater than 5.5:1 at an olefin feed weight hourly space velocity (WHSV) of 2 (g olefin/g catalyst/hr), (ii) greater than 4.5:1 at an olefin feed weight hourly space velocity of 3 (g olefin/g catalyst/hr), (iii) greater than 2.75:1 at an olefin feed weight hourly space velocity of 5 (g olefin/g catalyst/hr), or (iv) greater than 3.0:1 at an olefin feed weight hourly space velocity of 7 (g olefin/g catalyst/hr). Element 14: wherein the isomerization zeolite catalyst comprises a hydrogenation-active component. Element 15: wherein the hydrogenation-active component is palladium. Element 16: wherein a molar ratio of the at least one olefin to the hydrogen feed during the co-feeding step is between about 1:0.01 to about 1:1. Element 17: wherein the at least one olefin is isobutylene and the at least one skeletal isomer product is 1-butene and 2-butene. Element 18: wherein the at least one olefin comprises 1-butene and 2-butene, and the at least one skeletal isomer product is isobutylene. Element 19: wherein the isomerization zeolite catalyst comprises a hydrogenation-active component. Element 20: wherein the hydrogenation-active component is palladium. Element 21: wherein the catalyst cycle is at least sixteen days.

While certain embodiments have been shown and described, modifications thereof can be made by one skilled in the art without departing from the teachings of this disclosure. Numerous other modifications, equivalents, and alternatives will become apparent to those skilled in the art once the above disclosure is fully appreciated. It is intended that the following claims be interpreted to embrace such modification, equivalents, and alternatives where applicable.

The following references are incorporated by reference in their entirety for all purposes.
EP Pat. No. 0545179.
Atlas of Zeolite Structure Types” by W. M. Meier and D. H. Olson, Butterworths, 2nd Edition, 1987.
Collett and McGregor, Things go better with coke: the beneficial role of carbonaceous deposits in heterogeneous catalysis, Catal. Sci. Technol., 2016, 6, 363-378.
Guisnet et al., Skeletal Isomerization of n-Butenes, J. of Catalysis 158, 551-560 (1996).

1. A skeletal isomerization process comprising the steps of:
a) co-feeding a hydrocarbon feed comprising at least one olefin and a hydrogen feed to a reactor containing an isomerization zeolite catalyst; and
b) isomerizing the at least one olefin to at least one skeletal isomer product in the reactor for at least one catalyst cycle.

2. The skeletal isomerization process of claim 1, further comprising the step of recovering the at least one skeletal isomer product from the reactor.

3. The skeletal isomerization process of claim 1, wherein the hydrocarbon feed is fed at a weight hourly space velocity (WHSV) between 1 to 30h⁻¹.

4. The skeletal isomerization process of claim 1, wherein the isomerization zeolite catalyst is the hydrogen form of ferrierite (H-FER).

5. The skeletal isomerization process of claim 1, wherein a molar ratio of the at least one olefin in the hydrocarbon feed to the hydrogen feed during the co-feeding step is between about 1:0.01 to about 1:1.

6. The skeletal isomerization process of claim 1, wherein a molar ratio of the hydrocarbon feed to the hydrogen feed during the co-feeding step is between about 1:0.01 to about 1:1.

7. The skeletal isomerization process of claim 1, wherein the ratio of the hydrocarbon feed to the hydrogen feed is between about 2.5 vol. % and up to 50 vol. %, based on the volume of the total feed.

8. The skeletal isomerization process of claim 1, wherein the at least one olefin is an iso-olefin.

9. The skeletal isomerization process of claim 1, wherein the at least one olefin is isobutylene and the at least one skeletal isomer product is 1-butene and 2-butene.

10. The skeletal isomerization process of claim 1, wherein the at least one olefin comprises 1-butene and 2-butene, and the at least one skeletal isomer product is isobutylene.

11. The skeletal isomerization process of claim 1, wherein a temperature of the reactor is from about 340° C. to about 500° C.

12. The skeletal isomerization process of claim 1, wherein a pressure of the reactor is from zero to about 345 kPa (50 psig).

13. The skeletal isomerization process of claim 1, wherein the hydrocarbon feed comprises at least 40 wt. % isobutylene.

14. The process according to claim 1, wherein the ratio of time on stream for the at least one olefin conversion to reach 45% to linear butene (nB) yield is: (i) greater than 5.5:1 at an olefin feed weight hourly space velocity (WHSV) of 2 (g olefin/g catalyst/hr), (ii) greater than 4.5:1 at an olefin feed weight hourly space velocity of 3 (g olefin/g catalyst/hr), (iii) greater than 2.75:1 at an olefin feed weight hourly space velocity of 5 (g olefin/g catalyst/hr), or (iv) greater than 3.0:1 at an olefin feed weight hourly space velocity of 7 (g olefin/g catalyst/hr).

15. The process according to claim 1 wherein the isomerization zeolite catalyst comprises a hydrogenation-active component.

16. The process according to claim 1 wherein the hydrogenation-active component is palladium.

17. A skeletal isomerization process comprising the steps of:
a) co-feeding a hydrocarbon feed comprising at least one olefin and a hydrogen feed to a reactor containing an isomerization zeolite catalyst, wherein the hydrocarbon feed is fed at a weight hourly space velocity (WHSV) between 1 to 30 h⁻¹ and a molar ratio of the hydrocarbon feed to the hydrogen feed is between about 1:0.01 to about 1:1; and
isomerizing the at least one olefin to at least one skeletal isomer product in the reactor for at least one catalyst cycle, wherein the catalyst cycle is at least sixteen days, a
temperature of the reactor is from about 340° C. to about 500° C., and the isomerization zeolite catalyst is the hydrogen form of ferrierite (H-FER).

18. The skeletal isomerization process of claim 17, further comprising the step of recovering the at least one skeletal isomer product from the reactor.

19. The process according to claim 17 the ratio of time on stream for the at least one olefin conversion to reach 45% to linear butene (nB) yield is: (i) greater than 5.5:1 at an olefin feed weight hourly space velocity (WHSV) of 2 (g olefin/g catalyst/hr), (ii) greater than 4.5:1 at an olefin feed weight hourly space velocity of 3 (g olefin/g catalyst/hr), (iii) greater than 2.75:1 at an olefin feed weight hourly space velocity of 5 (g olefin/g catalyst/hr), or (iv) greater than 3.0:1 at an olefin feed weight hourly space velocity of 7 (g olefin/g catalyst/hr).

20. The process according to claim 17 wherein the isomerization zeolite catalyst comprises a hydrogenation-active component.
